(19) United States
(12) Patent Application Publication
(10) Pub. No.: US 2024/0172610 A1
(43) Pub. Date: May 30, 2024

(54) INTELLIGENT IRRIGATION DECISION SUPPORT SYSTEM

(71) Applicant: WAYCOOL FOODS AND PRODUCTS PRIVATE LIMITED, Chennai (IN)

(72) Inventors: Gopalakrishnan K A, Bengaluru (IN); Supriya ANANTHAKRISHNAN, Bengaluru (IN)

(21) Appl. No.: 18/281,133

(22) PCT Filed: Mar. 8, 2022
(86) PCT No.: PCT/IN2022/050203

§ 371 Date: Sep. 8, 2023

(30) Foreign Application Priority Data
Mar. 8, 2021 (IN) 202121009686

(51) Int. Cl. A01G 25/16

(52) U.S. Cl. CPC A01G 25/167; A01G 25/165

(57) ABSTRACT

An irrigation decision support system that integrates IOT (Internet of Things), artificial intelligence and user defined zone details for improving agricultural yield. The system factors in and monitors microclimatic conditions, soil water balance and predicts water loss in the future for efficient irrigation planning by using robust machine learning models. The user-defined zone inputs include factors like crop type, old irrigation, old depletion, future rainfall, soil profile and many other factors and provides recommendations to farmers via a mobile application.

### FIELD OF INVENTION

The current invention relates to an intelligent irrigation decision support system that integrates IOT (Internet of Things) and Artificial Intelligence for improving agricultural yield while optimizing resource utilization for agriculture. The system factors in and monitors microclimatic conditions, soil water balance and predicts crop water loss in the future for efficient irrigation planning by using robust machine learning models.

### BACKGROUND

Water and weather are the most critical aspects that impact the yield of crops. Infrequent rainfall, changes in temperature and unforeseen disease outbreaks lead to loss of harvesting, and reduction of yield for farmers. Modern agriculture demands high crop yield and quality while conserving water and energy. Hence there is a need to get accurate micro-climatic weather conditions for irrigation planning that can not only help improve the yield, but also optimize water utilization.

Most of the previous work done on irrigation management does not involve use of Machine Learning or analytics. Since agricultural data is seasonal in nature (time-series), these solutions focus more on finding patterns in historic data so as to give a general idea of how much water loss to expect at a certain time of the year. Though this may provide data with reasonable accuracy, but it is not totally accurate, due to the differences in such data between different seasons and crop cycle.

Some of the previously known irrigation management solutions which apply Machine Learning (ML), give water recommendations attained by exploiting only the past data. They do not anticipate future parameters like expected rainfall, which plays a vital role in deciding whether to irrigate on that day or next day, or not. Another limitation of these solutions is that these models do not factor in the soil water balance. Factoring in the soil water balance implies that once irrigated, a farm may not require any more irrigation for a few days. Hence daily irrigation in these solutions is in turn creating more stress on the agricultural land.

In addition, many solutions use already predicted meteorological weather data in computing future irrigation requirements. Predicted weather conditions themselves have inherent errors involved, and computing Evapotranspiration on these factors, can lead to further error in irrigation requirement prediction.

The method for irrigation management disclosed herein takes into account past localized parameters as well as future parameters before calculating the current water requirement.

The current invention discloses an intelligent irrigation decision support system that provides farmers with micro-climatic conditions of their farms, real time weather insights, and irrigation planning, so that they can take accurate, proactive, preventive decisions. The sensors installed on the farmer field continuously monitor the critical weather and soil parameters. In addition, weekly NDVI (Normalized Difference Vegetation Index) data from the satellite, provides information on the farm condition, soil moisture levels across the farm. The unique algorithm takes into account these Microclimatic conditions, satellite data, site specific information and provides notifications to the farmers or farming companies to take accurate, preventive actions. While most of the current irrigation solutions focus on the daily irrigation requirements and planning, the ML based prediction model included in the current invention helps in predicting the irrigation requirement for the upcoming future. This can not only help farmers plan their resources such as water, it significantly increases the yield at the end of crop cycle and prevents leaching of micronutrients and nitrogen.

### BRIEF DESCRIPTION OF FIGURES

FIG. 1: Shows Block Diagram of the Irrigation Decision Support System

FIG. 2: Shows various inputs provided to the AI module

FIG. 3: Shows Process flow for the steps for intelligent irrigation decision support system

FIG. 4: Shows Representation of Timing to irrigate as per Soil water balance model

FIG. 5: Shows Monthly Irrigation comparison between Historical irrigation and irrigation recommendation from Irrigation Decision Support System

FIG. 6: Shows Weekly Irrigation comparison between Historical irrigation and irrigation recommendation from Irrigation Decision Support System

FIG. 7: Shows daily variation of temperature captured by the IoT device

FIG. 8: Shows daily variation of humidity captured by the IoT device

FIG. 9: Shows daily variation of wind speed captured by the IoT device

FIG. 10: Shows daily variation of rainfall captured by the IoT device

### DETAILED DESCRIPTION

The current invention encompasses an intelligent irrigation decision support system that integrates IOT (Internet of Things), AI (Artificial Intelligence) along with satellite data monitoring and analysis for improving agricultural yield while optimizing resource utilization. The system factors in and monitors microclimatic conditions, soil water balance and predicts crop water loss in the future for efficient irrigation planning by using robust machine learning models.

While existing solutions use already predicted meteorological weather data (i.e. weather forecast) in computing future irrigation requirements. Predicted weather conditions themselves have inherent errors, and computing evapotranspiration on these factors, can lead to further error in irrigation requirement prediction or precise irrigation forecast.

### Definitions

As used herein, the term “Agro-climatic zone” is a land unit in terms of major climates, suitable for a certain range of crops and cultivars. The Planning Commission, as a result of the mid-term appraisal of the planning targets of the Seventh Plan, has divided the country (India) into fifteen broad agro-climatic zones based on physiography, soils, geological formation, climate, cropping patterns, and development of irrigation and mineral resources for broad agricultural planning and developing future strategies.

As used herein, the term “Microclimatic conditions” refers to the climatic conditions of the farm, where the device is installed, and the app is being used. Since climate can vary spatially, microclimate is referred to the climatic conditions of a small area.

Key microclimatic conditions include Temperature, Pressure, Humidity, Rainfall, Wind Speed and Wind Direction. Temperature, pressure, humidity are measured every 10 minutes while rainfall, windspeed, wind direction are measured continuously

These are the major microclimatic variables that are taken into consideration while providing an input to our ML model. Internally, there are many other derived features/variables that are calculated from these base weather inputs to improve our model's accuracy. These extra derived features not only contribute to the model's accuracy, but also allow us to generate valuable insights or conduct study for our understanding of the crop growth in that particular location.

As used herein, the term “zone” or “user defined zone” refers to a plot of land in a farmer's farm registered on the mobile app or any such user interface. Non Limiting examples of a user interface include any PC, desktop, mobile handheld device, smartphones. Users can add crop type, soil type, crop acreage, irrigation type, sowing date etc for the crops grown in each zone and they get water recommendations separately for each of their zones. There are no limits on the number of zones that a user can create.

As used herein, the term “Artificial Intelligence module” refers to use of machine learning, deep learning or neural networks or a combination thereof to model one or more of micro climatic conditions, agroclimatic zone aspects, soil moisture balance, satellite data or a combination thereof.

As used herein, “Evapotranspiration” (ETo) refers to water loss emanating from the sum of evaporation from land surface and transpiration from plants. Several environmental factors like Solar Radiation, Vapor Pressure Deficit, Solar Declination etc affect the rate of evapotranspiration. As used herein, “Reference Evapotranspiration” refers to the implementation of FAO56 Penman based Evapotranspiration. As used herein “Predicted Evapotranspiration” is computed by the artificial intelligence module.

As used herein, the term “soil water balance model” is defined as an irrigation scheduling approach/system to keep track of the soil water deficit by accounting for the amount of water entering, exiting the root zone of the plant in a given period. The sources of incoming water can be irrigation, rainfall, subsurface inflow etc while the sources of exiting water can be evapotranspiration, soil run-off, percolation and many other similar factors.

As used herein, the term “soil moisture threshold” is defined as the point below which a plant finds it difficult to extract water from the root system and hence is considered as the refill point/irrigation point. This is the point from where if the water is not provided to the plant, stomata begin to close and the plant becomes more prone to withering. Soil moisture threshold is determined using the soil water balance model.

As used herein, the term “Cumulative depletion” is computed by factoring in previous day depletion, current ETo and rainfall and irrigation.

Hence depletion and cumulative depletion is dependent on ETo, rainfall, irrigation. Other factors such as soil runoff can be considered as well.

As used herein, the “FAO PM 56” or “FAO-56 PM” or “FAO-56 Penman method” are used interchangeably herewith and refer to the Penman-Monteith method adopted by the Food and Agriculture Organization (FAO) in its Irrigation and Drainage Paper No. 56. Known as FAO 56 PM, this method is a global standard based on meteorological data (Allen RG, Jensen ME, Wright JL, Burman RD. 1989. Operational estimates of reference evapotranspiration. Agronomy Journal 81(4):650-662), and it has been found to work well in numerous locations if the required data are available. The FAO 56 PM method requires measurements of temperature, relative humidity, wind speed, solar radiation and many other factors. This data demand is the main constraint on its use in locations where climate data are limited. This is a common problem in developing countries and especially for tropical regions and high-altitude areas.

As used herewith “irrigation forecast” constitutes the volume of water and date of irrigation. It is also referred as “irrigation recommendation” or “irrigation requirement”

### Embodiments

The invention is to be understood as not being limited to particular embodiments described, as such may, of course, vary. It is also to be understood that the terminology used herein is for the purpose of describing particular embodiments only, and is not intended to be limiting, since the scope of the present invention will be limited only by the appended claims.

In one embodiment, intelligent irrigation decision support system, the system comprising;
an IoT device (100) equipped with a plurality of on ground sensors to gather data on microclimatic conditions;
a cloud computation unit (101) configured with an artificial intelligence module (102) and wherein the artificial intelligence module creates a plurality of models trained on historic weather data specific to agroclimatic zones;
a user interface (103) to gather user defined zone inputs from a user-optionally an automated pump (104) coupled to the computation unit; a communication hub (105) configured to connect the IoT device (100), the cloud computation unit (101), the user interface (103) and optionally the automated pump through wired or wireless means; and
wherein the artificial intelligence (102) module inputs the data received from the IoT device to at least one of the plurality of models trained on historic weather data specific to agroclimatic zones to predict future evapotranspiration and outputs an irrigation forecast after processing inputs from the user interface (103) and factoring in a soil water balance model.

FIG. 1 illustrates the architecture of the Intelligent irrigation decision support system of the invention. The IoT device (100) is equipped with a plurality of sensors that gather data on microclimatic conditions of a farm or a field. The gathered data is transmitted to the Cloud computation unit (101) configured with an Artificial Intelligence module (102). The cloud computation unit (101) received inputs from the User interface (103), the inputs gathered include user defined zone inputs. The communication hub (105) is communicatively coupled with the IoT device (100), Cloud computation unit (101), User interface (103) as well as a pump (104) through wired or wireless means.

In one embodiment, the user interface (103) is communicatively coupled with the cloud computation unit (101).

The communication hub (105) facilitates data transmission and exchange.

In one embodiment the artificial intelligence module deploys a machine learning model to predict future evapotranspiration based on historic weather data of at least 40 years specific to every agroclimatic zone. In one embodiment, the machine learning model further takes input from the IOT device, on a daily basis to improve the prediction.

In one embodiment, the AI module deploys machine learning models selected from, without limitation, decision tree, Random Forest, Adaptive Boosting, Gradient Boosting Machine, Extreme Gradient Boosting (XGBoost), K-Nearest neighbours algorithm, Naïve Byes, deep learning, linear or logistic regression, K means clustering. The machine learning models can be a combination of classification, clustering and regression models. In one embodiment, the machine learning models were selected on the basis of weighted standard error of estimate (WSEE). In one embodiment a WSEE value of less than 1 mm is ideal for selection of the model.

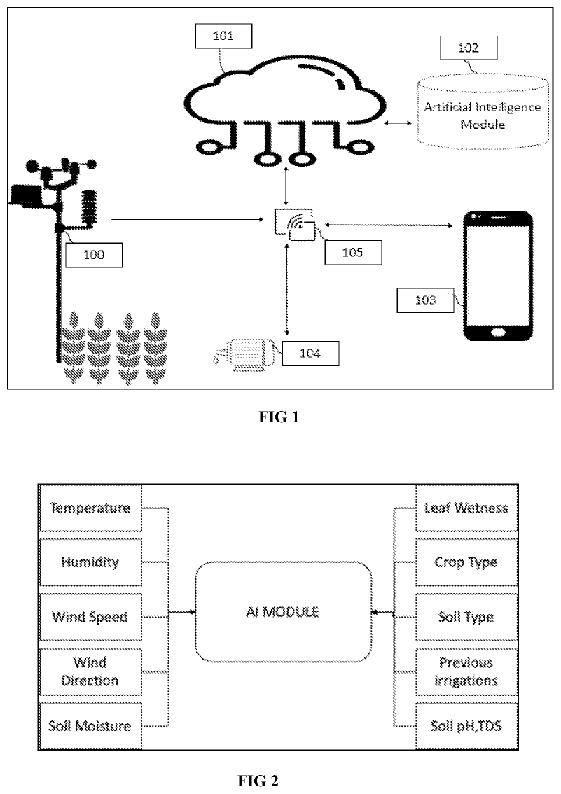

FIG. 2 depicts the parameters input to the Artificial Intelligence (102) or AI module.

In one embodiment, the historic weather data specific to the agroclimatic zones is gathered over at least 40 years.

In one embodiment of the invention the irrigation forecast includes irrigation frequency and volume of water for each zone.

In one embodiment of the invention, the IoT device (100) equipped with a plurality of on ground sensors further comprises a Central module which is a part of system hardware that controls the operation of sensors. In one embodiment, the central module comprises a microcontroller, a battery and the module runs and controls the sensors. This module is deployed on the field with the plurality of sensors that are equipped with the IoT device. In one embodiment the IoT module has sleep modules that enable battery optimization.

In one embodiment of the invention, the IoT device is a standalone device. In one embodiment of the invention, the IoT device operates on solar energy.

In one embodiment of the invention the IoT device operates on renewable energy.

In one embodiment of the invention the IoT device operates on electricity or biofuel or solar energy or a combination thereof.

In one embodiment, the plurality of on ground sensors of the IoT device (100) sense microclimatic conditions selected from wind speed, humidity, temperature, leaf wetness, wind direction, soil pH, soil TDS, electrical conductivity or a combination thereof.

In one embodiment of the invention the on-ground sensors can be connected to a central module, powered by solar energy.

In one embodiment of the invention the data from the sensors is sent to the central module comprising a microcontroller with signal conditioning circuits. The data is then sent to the cloud computation unit using a GSM module. The entire module entails a unique architecture which is modular in fabrication.

In one embodiment of the invention, the IoT device (100) has a memory and stores the sensed values of the microclimatic conditions in a database. In one embodiment of the invention, the IoT device (100) transmits the sensed values of the microclimatic conditions to the cloud computation unit on an hourly basis.

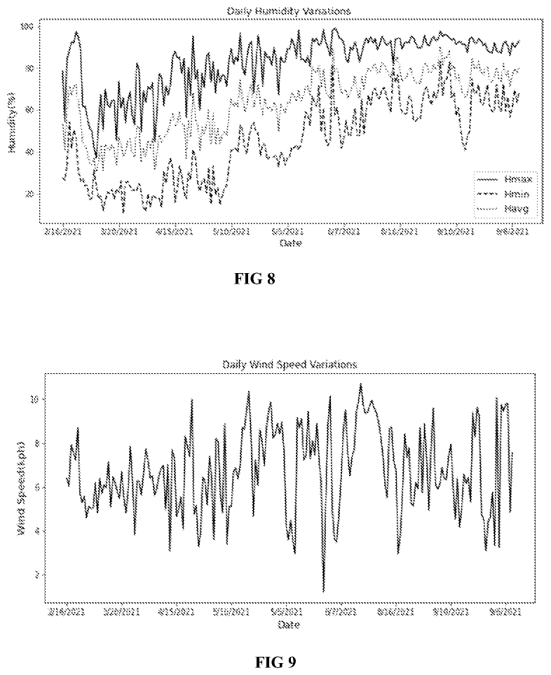

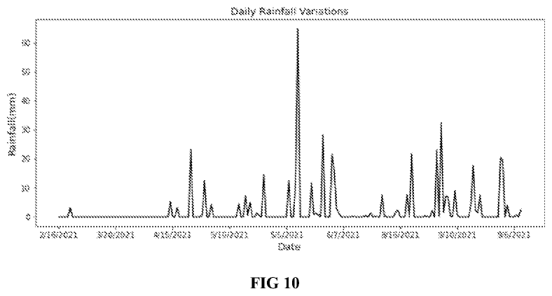

FIGS. 7,8,9,10 depict various microclimatic conditions (temperature, humidity, wind speed and rainfall respectively) captured by the IoT device and the variation therein.

In one embodiment the IoT device can be deployed in an area range of from 5-50 hectares of land. In one embodiment of the invention, the soil moisture sensors of the IoT device captures the soil conditions for the land where it is installed.

In one embodiment of the invention the cloud computation unit of the irrigation planning system receives and processes Satellite data. In one embodiment of the invention the satellite data is transformed to generate NDVI (Normalized Difference Vegetation Index), NDWI (Normalized Difference Water Index), EVI (Enhanced Vegetation Index), soil moisture.

In one embodiment of the invention, the user defined zone inputs gathered by the user interface (103) comprise of farm size, crop type, soil type, sowing date/time, pump type, and irrigation method used.

In one embodiment of the invention the data gathered from a zone is stored in the cloud/back end.

In one embodiment of the invention the irrigation system of the invention provides an irrigation forecast for at least 5-7 days in advance.

In one embodiment of the invention, the artificial intelligence module (102) configured with the cloud computation unit (101) inputs the data gathered on microclimatic conditions by the IoT device (100) to the model trained on historic weather data specific to agroclimatic zones on a daily basis.

In one embodiment of the invention, for ETo prediction model the derived values of minimum and maximum temperature, minimum and maximum humidity sensors of the IoT device (100), as well as the last 7 days of reference ETo values deriving from micro climatic conditions along with the rolling mean of last 7 days of ETo values are considered.

In one embodiment of the invention, the data gathered on microclimatic conditions by the IoT device is used to compute reference evapotranspiration based on FAO-56 PM method. In one embodiment of the invention, the reference evapotranspiration is computed on a daily basis by the cloud computation unit. In one embodiment, the computed values of reference evapotranspiration provide feedback to the plurality of models created by the artificial intelligence module based on historic weather data. In one embodiment, the computed values of reference evapotranspiration provide feedback to at least one of the plurality of models trained on historic weather data specific to agroclimatic zones.

In one embodiment of the invention, the artificial intelligence module (102) configured with the cloud computation unit creates a plurality of models based on the historic data for agroclimatic zones.

In one embodiment, the artificial intelligence module (102) configured with the cloud computation unit (101) inputs the data gathered on microclimatic conditions by the IoT device (100) to the model trained on historic weather data specific to agroclimatic zones. In one embodiment the on a daily basis artificial intelligence module (102) configured with the cloud computation unit (101) inputs the data gathered on microclimatic conditions by the IoT device (100) to the model trained on historic weather data specific to agroclimatic zones on a daily basis.

### Initial Data Processing—Computation of Daily Evapotranspiration

Daily potential evapotranspiration is computed daily using penman-monteith (P-M) method which is described below.

$$0.408 Δ ( {R}_{n} - G ) + γ \frac{900}{( T + 273 )} {u}_{2} ( {e}_{a} - {e}_{d} ) ETo + \frac{mean}{Δ + {γ}^{*}}$$

Where,
ETo=Reference Evapotranspiration, [mm/d]
Δ=Slope of the saturation vapor pressure-temperature curve, [kPa° C.⁻¹]
Rₙ=Net Radiation, [MJ m⁻² d⁻¹]
G=Solar Heat flux density to the ground, [MJ m⁻² d⁻¹]
γ=Psychrometric constant, [kPa° C.⁻¹]
Tₘₑₐₙ=Mean of maximum and minimum temperature, [° C.⁻¹]
u₂=Horizontal wind speed at 2 meter height, [m/s]
eₐ=Saturation vapor pressure at air temperature, [kPa]
eₐ=Saturation vapor pressure at air temperature, [kPa]

In one embodiment of the invention, the model is trained using historic climatic data to provide site specific insights about seasonality in climate change, crop-based, or stage of growth-based cultivation practices or a combination thereof. In one embodiment of the invention the actual reference ETo is computed daily to fine tune predictions made by artificial intelligence modules. In one embodiment of the invention the artificial intelligence module models the soil water balance.

In one embodiment, the data from the sensed microclimatic conditions sensed by the plurality of sensors of IoT device (100) is used to compute reference evapotranspiration or reference ETo. In one embodiment the reference ETo is computed daily.

In one embodiment, the soil water balance takes into account current soil water level, present time or current evapotranspiration and any rainfall, irrigation values. This computation is done for the future days also, with predicted evapotranspiration. In one embodiment the soil water balance model determines the soil water threshold value.

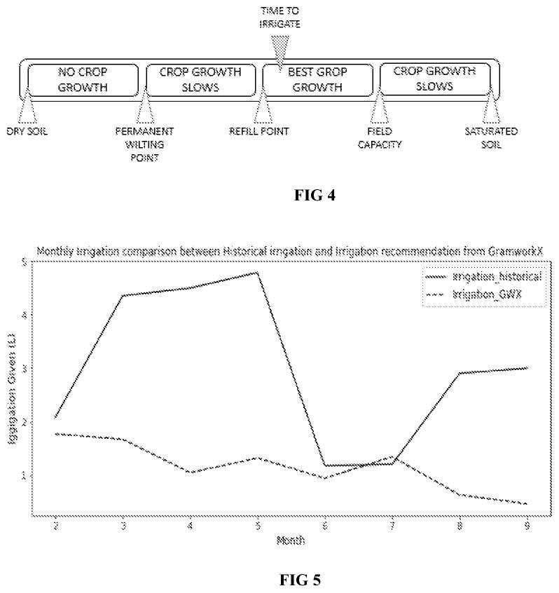

FIG. 4 depicts the Timing of irrigation based on the soil water balance model. Refill point is indicated when depletion cumulative is greater or equal to the soil moisture threshold. In one embodiment the timing of irrigation is indicated to users on a user interface.

In one embodiment of the invention the cloud computation unit sends an output to the user interface wherein the output comprises the irrigation forecast. In one embodiment of the invention, the output includes constant in-app reminders to irrigate. In one embodiment of the invention, the output includes constant in-app reminders for 2 days continuously to irrigate. The irrigation forecast comprises frequency (irrigation date) and volume of water to be used. In one embodiment of the invention the irrigation forecast is dependent upon the soil type, nature of the crop, past irrigation/depletion or a combination thereof. In one embodiment of the invention the irrigation forecast is in the form of an alert.

In one embodiment of the invention the irrigation forecast comprises volume of water, and timing of irrigation for a user defined zone.

In one embodiment of the invention the irrigation forecast factors in the soil water balance.

In one embodiment the user interface includes any human machine interface and includes, without limitation, user interfaces on website, phone, computer, smart phone, tablets, mobile application, mobile handheld devices, desktops.

In one embodiment of the invention the computing unit sends an output to the automated pump wherein the output comprises the irrigation requirement. as determined based on the irrigation forecast. The irrigation-requirement comprises frequency and/or volume of water to be used in a zone. In one embodiment of the invention the irrigation forecast is dependent upon the soil type and nature of the crop. In one embodiment of the invention the irrigation forecast factors in the soil water balance.

In one embodiment of the invention the automated pump can be turned off or on remotely. In one embodiment of the invention the pump can be turned on or off via the mobile application or web-based dashboard.

In one embodiment of the invention, based on the irrigation forecast or recommendation provided in liters and knowing the motor release in liter/hour, the farmer can directly control the motor from the mobile/web app and automatically turn the pump on/off. In one embodiment, the analytic processing of the sensor inputs along with derived features is done by the artificial intelligence module to predict water loss in future.

In one embodiment, the current invention also accounts for the soil water balance model which takes into account calculated water loss, old rainfall pattern, previous irrigation, old depletion, future expected rainfall and many more such parameters to provide on-field water requirements up to a week.

In one embodiment, the current invention encompasses pump automation that directly takes feed from the recommendations given to farmers.

One embodiment of the invention is method of computing future evapotranspiration and determining irrigation forecast for a user defined zone, the method comprising the steps of;
a) gathering data on microclimatic conditions by an IoT device (100) equipped with a plurality of on-ground sensors;
b) transmitting the data gathered by the plurality of on ground sensors of the IoT device on the microclimatic conditions to a cloud computation unit (101) configured with an artificial intelligence module (102) and wherein the artificial intelligence creates a plurality of models trained on historic weather data specific to agroclimatic zones;
c) prediction of the future evapotranspiration value by the artificial intelligence module (102) based on modelling the historic data for agroclimatic zones and adding inputs of the data gathered on microclimatic conditions sensed by the plurality of on ground sensors on the IoT device (100) to at least one of the models created in step b);
d) integrating the data inputs from a user defined zone by a user interface (103) with the predicted future evapotranspiration value in step c)
and wherein the AI module factors in a soil water balance model to output an irrigation forecast.

In one embodiment, the artificial intelligence module (102) inputs the data gathered on microclimatic conditions by the IoT device (100) to at least one of the plurality of models trained on historic weather data specific to agroclimatic zones on a daily basis in step c).

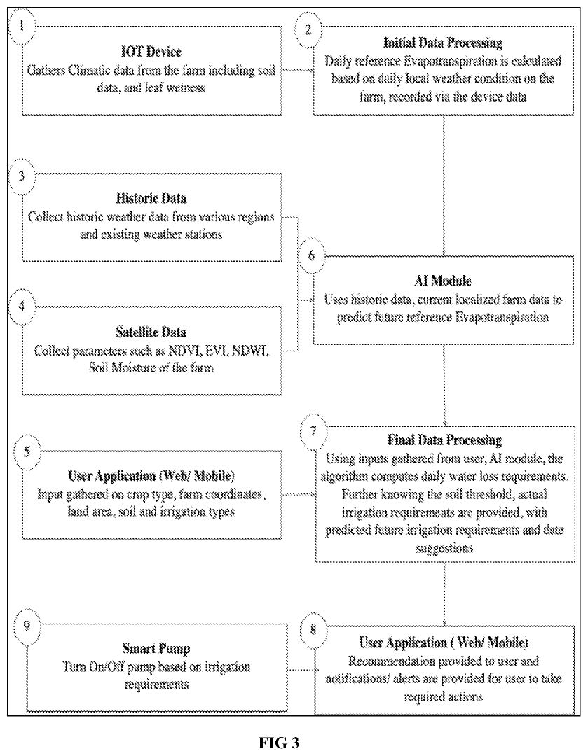

FIG. 3 depicts the workflow of the Intelligence Irrigation decision support system of the invention.

In one embodiment, the data inputs gathered on microclimatic conditions by the IoT device (100) are fed to the model trained on historic weather data specific to agroclimatic zones on an hourly basis to predict future evapotranspiration in step c).

In one embodiment, the soil water balance model factors in soil moisture threshold value for a user defined zone.

In one embodiment, the irrigation decision support system of the invention provides per plant irrigation requirements for drip irrigation.

### EXAMPLES

### Example 1: Modelling Historical Data, Evapotranspiration Prediction of ETo

Historic climatic data was used to provide insights about seasonality in climate change, crop-based, and stage of growth-based cultivation practices.

### 1: Future ETo Prediction:

a. Weather data for Last 40 years has been taken from a given agroclimatic zone to train models. These models learn the inherent patterns and correlations present in the data and use these learned correlations to predict ETo for today based on on-field sensor data as captured by the IoT device (100)
b. 80% data was used to train the model and 20% to test the model and model's performance was evaluated on different batches of test data to ensure robustness on unseen data
c. Reference ETo calculated using FAO56 Penman method is constantly used to fine-tune the above said models.

Five different type of machine learning model namely (i) decision tree, (ii) Random Forest (RF), (iii) Adaptive Boosting (AdaBoost), (iv) Gradient Boosting Machine (GBM) and (v) Extreme Gradient Boosting (XGBoost), were used to estimate the P-M ETo. These learning models were developed using the daily records basic meteorological data for the period 1971-2017 and were tested for 2018-2020.

The best model was selected on the basis of weighted standard error of estimate (WSEE). The RF, GBM and XGBoost model performed extremely well as WSEE values for these models were 0.245, 0.206 and 0.166 mm/d respectively. It is concluded that the ensemble machine learning model substantiated by Extreme Gradient Boosting significantly enhances the performance in estimating P-M ETo. Moreover, the sensitivity analysis suggested that the data requirement for XGBoost is commonly available at most of the places unlike the P-M ETo model. Though the model was developed for the Southern plateau and hill region of Indian sub-continent (Agro-ecological zone 10), given the generalization capability of the model, it can be successfully implemented for other similar locations where comprehensive data are not available.

### Initial Data Processing—Computation of Daily Evapotranspiration

Daily potential evapotranspiration is computed daily using penman-monteith (P-M) method which is described below.

$$0.408 Δ ( {R}_{n} - G ) + γ \frac{900}{( T + 273 )} {u}_{2} ( {e}_{a} - {e}_{d} ) ETo + \frac{mean}{Δ + {γ}^{*}}$$

Where,
ETo=Reference Evapotranspiration, [mm/d]
Δ=Slope of the saturation vapor pressure-temperature curve, [kPa ° C.⁻¹]
Rₙ=Net Radiation, [MJ m⁻² d⁻¹]
G=Solar Heat flux density to the ground, [MJ m⁻² d⁻¹]
γ=Psychrometric constant, [kPa ° C.⁻¹]
Tₘₑₐₙ=Mean of maximum and minimum temperature, [° C.⁻¹]
u₂=Horizontal wind speed at 2 meter height, [m/s]
eₐ=Saturation vapor pressure at air temperature, [kPa]
e_{d}=Saturation vapor pressure at air temperature, [kPa]

### Example 2: Irrigation Forecast and Water Savings

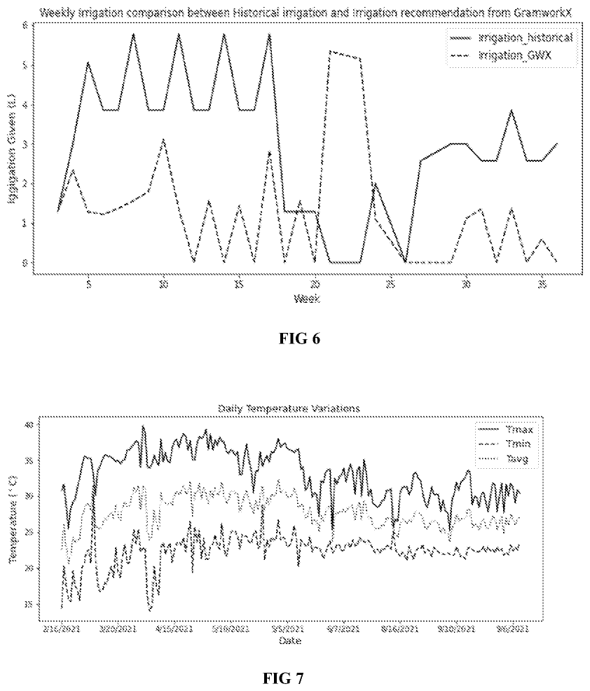

FIG. 5 and FIG. 6 graphs indicate the historic irrigation done by the farmer for the crop in the same stage (by touch and feel method) Vs the Irrigation recommendation provided by the Intelligent irrigation decision support system of the invention.

The “Touch and feel method” is where the user/farmer check the soil, with hand and feels the amount of soil moisture. This is a very rudimentary and unscientific way to assess soil moisture.

There is a cumulative irrigation savings of 448 L per plant in the farm and the total water savings in the farm is 1612800 L compared to the historical irrigation done by the farmer.

### Example 3: Role of Soil Moisture Threshold in Irrigation Forecast

For Grape plants growing in 3.6 acres (one crop zone), and in Shoot growth stage, with a crop factor (Kc) of 0.4. The role of soil moisture threshold and irrigation forecast is calculated as below.

The cloud computation unit, takes into account, based on the crop type, sowing date and irrigation type, the crop and stage specific Crop Factor and irrigation efficiency.

The crop factor varies in different stages of the plant growth. Examples of stages of grapes are—Fruit pruning, shoot growth, bloom etc.

Soil Moisture threshold for this plant is calculated by knowing the type of Soil and crop root depth. For example if the Soil Type is Clay Loam and crop root depth is 0.5 meter, the soil moisture threshold is 12 mm and calculated using the below formulae:

SWHC=FC−WP (vol %)

TAW=SWHC*Rd

RAW=p*TAW

Soil Water Threshold=x %*RAW
Where,
   SWHC=Soil Water Holding Capacity
   FC=Field Capacity
   WP—Wilting Point
   TAW=Total Available Water
   Rd=Root Depth
   RAW=Readily Available Water, p=fraction of TAW
   Soil Water Threshold—is a % of RAW, that can be varied from crop to crop, based on their irrigation criticality.

Scenario 1: As seen below in Table 1, as the cumulative depletion crosses Soil Moisture Threshold, 6 days from today, indication is given to the user to irrigate. In this scenario there is no future rainfall or other expected irrigation.

The amount of water loss today for grapes zone of 3.6 acres is 4.05 mm, Then the irrigation requirement is no irrigation requirement today.

Irrigation will be required 6 days from now, since cumulative depletion in soil will cross the soil moisture threshold. Cumulative depletion 6 days from now will be 12.69 mm. Total irrigation required for 3.6 acres will be 184,837.4 L or per plant requirement will be (12.69 mm*area of plant root)Ls 6 days from now

TABLE 1

| | | | ETc − Crop | | | | Soil | |
|---|---|---|---|---|---|---|---|---|
| | | Predicted | Evapotranspiration = | | | Depletion | moisture | Irrigation |
| Day | ETo | ETo | ETo* Kc | Rainfall | Irrigation | cumulative | Threshold | Forecast |
| Today | 4.05 mm | | 1.62 mm | 0 mm | 0 mm | 1.62 mm | 12 mm | No |
| | | | | | | | | Irrigation |
| | | | | | | | | required |
| Tomorrow | | 4.25 mm | 1.7 mm | 0 mm | 0 mm | 3.32 mm | 12 mm | No |
| (Today + 1) | | | | | | | | Irrigation |
| | | | | | | | | required |
| Today + 2 | | 4.55 mm | 1.82 mm | 0 mm | 0 mm | 5.12 mm | 12 mm | No |
| | | | | | | | | Irrigation |
| | | | | | | | | required |
| Today + 3 | | 4.40 mm | 1.76 mm | 0 mm | 0 mm | 6.88 mm | 12 mm | No |
| | | | | | | | | Irrigation |
| | | | | | | | | required |
| Today + 4 | | 4.23 mm | 1.69 mm | 0 mm | 0 mm | 8.57 mm | 12 mm | No |
| | | | | | | | | Irrigation |
| | | | | | | | | required |
| Today + 5 | | 5.01 mm | 2.01 mm | 0 mm | 0 mm | 10.58 mm | 12 mm | No |
| | | | | | | | | Irrigation |
| | | | | | | | | required |
| Today + 6 | | 5.27 mm | 2.11 mm | 0 mm | 0 mm | 12.69 mm | 12 mm | Irrigation |
| | | | | | | | | required |
| Today + 7 | | 4.90 mm | 1.96 | 0 mm | 12.7 mm | 1.95 mm | 12 mm | Irrigation |
| | | | | | | | | not |
| | | | | | | | | required |

Scenario 2: As seen below in Table 2, as the cumulative depletion does not cross Soil Moisture Threshold, in the future 7 days, and hence there is no need for the farmer to irrigate, as in this scenario there is future rainfall expected.

TABLE 2

| | | | ETc − Crop | | | | Soil | |
|---|---|---|---|---|---|---|---|---|
| | | Predicted | Evapotranspiration = | | | Depletion | moisture | Irrigation |
| Day | ETo | ETo | ETo* Kc | Rainfall | Irrigation | cumulative | Threshold | requirement |
| Today | 4.05 mm | | 1.62 mm | 0 mm | 0 mm | 1.62 mm | 12 mm | No |
| | | | | | | | | Irrigation |
| | | | | | | | | required |
| Tomorrow | | 4.25 mm | 1.7 mm | 0 mm | 0 mm | 3.32 mm | 12 mm | No |
| | | | | | | | | Irrigation |
| | | | | | | | | required |
| Today + 2 | | 4.55 mm | 1.82 mm | 0 mm | 0 mm | 5.12 mm | 12 mm | No |
| | | | | | | | | Irrigation |
| | | | | | | | | required |
| Today + 3 | | 4.40 mm | 1.76 mm | 0 mm | 0 mm | 6.88 mm | 12 mm | No |
| | | | | | | | | Irrigation |
| | | | | | | | | required |
| Today + 4 | | 4.23 mm | 1.69 mm | 5 mm | 0 mm | 3.57 mm | 12 mm | No |
| | | | | | | | | Irrigation |
| | | | | | | | | required |
| Today + 5 | | 5.01 mm | 2.01 mm | 2 mm | 0 mm | 3.58 mm | 12 mm | No |
| | | | | | | | | Irrigation |
| | | | | | | | | required |
| Today + 6 | | 5.27 mm | 2.11 mm | 0 mm | 0 mm | 5.69 mm | 12 mm | No |
| | | | | | | | | Irrigation |
| | | | | | | | | required |
| Today + 7 | | 4.90 mm | 1.96 mm | 0 mm | 0 mm | 7.65 mm | 12 mm | Irrigation |
| | | | | | | | | not |
| | | | | | | | | required |

Advantages of predicted ETo and soil water balance is to prevent users from just irrigating every 2-5 days, as they usually do. This helps users to know when and how much to irrigate.

In the crop coefficient approach the crop evapotranspiration, ET_{c}, is calculated by multiplying the reference crop evapotranspiration, ETₒ, by a crop coefficient, K_{c}:

ET_{c}=K_{c}ETₒ (56)
where
ET_{c} crop evapotranspiration [mm d⁻¹],
K_{c} crop coefficient [dimensionless],
E_{T} reference crop evapotranspiration [mm d⁻¹].

ET_{c} is derived in the AI module, after the ETo is known. Crop details such as sowing date also are need to know the crop factor. Crop factor are standard.

Cumulative depletion=previous day depletion+ET_{c}(today)−rainfall−irrigation.

Hence depletion and cumulative depletion is dependent on ETo, rainfall, irrigation.

1. An intelligent irrigation decision support system, the system comprising; an IoT device equipped with a plurality of on ground sensors to gather data on microclimatic conditions;
a cloud computation unit configured with an artificial intelligence module and wherein the artificial intelligence module creates a plurality of models trained on historic weather data specific to agroclimatic zones;
a user interface to gather user defined zone inputs from a user; optionally an automated pump coupled to the computation unit;
a communication hub configured to connect the IoT device, the cloud computation unit, the user interface and optionally the automated pump through wired or wireless means; and
wherein the artificial intelligence module inputs the data received from the IoT device to at least one of the plurality of models trained on historic weather data specific to agroclimatic zones to predict future evapotranspiration and outputs an irrigation forecast after processing inputs from the user interface and factoring in a soil water balance model.

2. The system as claimed in claim 1 wherein the plurality of on ground sensors of the IoT device sense microclimatic conditions selected from wind speed, humidity, temperature, leaf wetness, wind direction, soil pH, soil TDS, electrical conductivity or a combination thereof.

3. The system as claimed in claim 1 wherein the user defined zone inputs gathered
by the user interface comprise of farm size, crop type, soil type, sowing date/time, pump type, and irrigation method used.

4. The system as claimed in claim 1 wherein the future evapotranspiration is predicted for 5-7 days in advance.

5. The system as claimed in claim 1 wherein the historic weather data specific to the agroclimatic zones is gathered over at least 40 years.

6. The system as claimed in claim 1 wherein the artificial intelligence module
configured with the cloud computation unit inputs the data gathered on microclimatic conditions by the IoT device to the model trained on historic weather data specific to agroclimatic zones on a daily basis.

7. The system as claimed in claim 1 wherein the data gathered on microclimatic
conditions by the IoT device is used to compute reference evapotranspiration based on FAO-56 PM method.

8. The system as claimed in claim 1 wherein the irrigation forecast comprises volume of water, and timing of irrigation for a user defined zone.

9. A method of computing future evapotranspiration and determining irrigation forecast for a user defined zone, the method comprising the steps of;
a) gathering data on microclimatic conditions by an IoT device equipped with a plurality of on ground sensors;
b) transmitting the data gathered by the plurality of on ground sensors of the IoT device on the microclimatic conditions to a cloud computation unit configured with an artificial intelligence module and wherein the artificial intelligence creates a plurality of models trained on historic weather data specific to agroclimatic zones;
c) prediction of the future evapotranspiration value by the artificial intelligence module based on modelling the historic data for agroclimatic zones and adding inputs of the data gathered on microclimatic conditions sensed by the plurality of on ground sensors on the IoT device to at least one of the models created in step b);
d) integrating the data inputs from a user defined zone by a user interface with the predicted future evapotranspiration value in step c) and wherein the AI module factors in a soil water balance model to output an irrigation forecast.

10. The method as claimed in claim 9 wherein the artificial intelligence module inputs the data gathered on microclimatic conditions by the IoT device to at least one of the plurality of models trained on historic weather data specific to agroclimatic zones on a daily basis in step c).

11. The method as claimed in claim 9 wherein the soil water balance model factors in soil moisture threshold value for a user defined zone.
